(19) United States
(12) Patent Application Publication
(10) Pub. No.: US 2024/0198133 A1
(43) Pub. Date: Jun. 20, 2024

(54) SYSTEM AND METHOD FOR INVERSE TREATMENT PLANNING FOR RADIATION THERAPY

(71) Applicants: The General Hospital Corporation, Boston, MA (US); Tilburg University, Tilburg (NL)

(72) Inventors: Thomas Bortfeld, Cambridge, MA (US); Ali Ajdari, Cambridge, MA (US); Stefan ten Eikelder, Tilburg (NL); Dick den Hertog, Tilburg (NL)

(21) Appl. No.: 18/549,697

(22) PCT Filed: Mar. 8, 2022
(86) PCT No.: PCT/US2022/019275

§ 371 Date: Sep. 8, 2023

Related U.S. Application Data
(60) Provisional application No. 63/158,193, filed on Mar. 8, 2021.

(51) Int. Cl. A61N 5/10; G06F 17/11

(52) U.S. Cl. CPC A61N 5/103; G06F 17/11

(57) ABSTRACT

Systems and methods are provided for determining a treatment plan for a radiation therapy system. The method includes dividing a three-dimensional volume of a patient into a grid of dose voxels, wherein at least a portion of said dose voxels are designated to belong to at least one target or to at least one critical structure. The method also includes modeling an ionizing radiation dose as delivered by a plurality of beamlets each having a beamlet fluence, to create a modeled radiation dose fluence map. The method also includes determining a voxel-based fluence map optimization (FMO) model for the modeled radiation dose a fluence map. The method also includes determining a conic optimization formulation for the FMO model to create a determined conic optimization solution and generating an optimized fluence map by updating the modeled radiation dose fluence map with the determined conic optimal fluence map.

### CROSS-REFERENCE TO RELATED APPLICATIONS

This application claims the benefit of U.S. Provisional Patent Application Ser. No. 63/158,193 filed on Mar. 8, 2021 and entitled “System and Method for Inverse Treatment Planning for Radiation Therapy,” which is incorporated herein by reference as if set forth in its entirety for all purposes.

### STATEMENT REGARDING FEDERALLY SPONSORED RESEARCH

N/A

### BACKGROUND

Fluence map optimization (FMO) is one of the core elements of inverse treatment planning for external beam radiation therapy (RT). It aims to find the combination of fluences (beamlet intensities) that yield the optimal trade-off between various treatment plan evaluation criteria. Since the earliest days of intensity modulated radiation therapy (IMRT), this has been accomplished in an inverse fashion, and the FMO has been automated.

In general, determining the optimal fluence map is a large-scale optimization problem and requires the use of advanced numerical solvers. The types of employed evaluation criteria determine whether the optimization problem is linear, quadratic, or otherwise convex or non-convex. For example, minimum, mean, and maximum dose criteria lead to linear functions, and quadratic errors from a prescription dose lead to quadratic functions. Dose-volume based constraints are generally known to be non-convex. Biologically-based evaluation criteria, such as tumor control probability (TCP) and normal tissue complication probability (NTCP) criteria often have more complicated structures involving exponentials, logarithms and power functions.

To solve these problems, commercial treatment planning systems (TPS) use a variety of solution methods. RayStation uses sequential quadratic programming, Varian Eclipse uses conjugate gradient methods, Philips Pinnacle uses a limited memory Broyden-Fletcher-Goldfarb-Shanno (L-BFGS) algorithm adapted to account for constraints, and Elekta Monaco uses an (interior point) barrier method. The CERR toolbox requires a user pre-installed solver, and the matRad toolbox calls the open-source general convex optimization solver IPOPT.

Next to this, many different solution approaches have been considered to improve speed and/or solution quality for specific problem formulations. Most of these solution approaches do not guarantee globally optimal solutions, nor do they recognize if such a solution has been found.

Treatment planning systems, both commercial and research versions, use a variety of different solution approaches to FMO problems. The majority are either restricted to the particular problem form considered, or do not guarantee optimal solutions to the FMO problem.

Thus, there is a need for fast, high-quality, fluence map solutions that yield the desired trade-off between various treatment plan evaluation criteria. There also remains a need for a globally optimal solution.

### SUMMARY OF THE DISCLOSURE

The present disclosure overcomes the aforementioned drawbacks by providing systems and methods for FMO with treatment plan criteria. In one non-limiting example, the treatment plan criteria may include using a conic optimization formulation. Conic optimization is a structured approach to mathematical optimization. The present disclosure recognizes that modeling FMO problems as conic optimization problems can enable the use of advanced numerical solvers for FMO problems, and result in faster and better solutions to FMO problems than conventional methods for FMO.

In one configuration, a method is provided for determining a treatment plan for a radiation therapy system. The method includes determining a three-dimensional region of interest (ROI) in a subject containing a critical structure, dividing the ROI into a grid of dose voxels, and modeling a radiation dose fluence map for the grid of dose voxels as delivered by the radiation therapy system with a plurality of beamlets to create a modeled radiation dose fluence map, each having a beamlet fluence. The method also includes determining a voxel-based fluence map optimization (FMO) model for the modeled radiation dose fluence map, determining a conic optimization formulation for the FMO model to create a determined conic optimization solution, and generating an optimized fluence map by updating the modeled radiation dose fluence map with the determined conic optimization solution.

In one configuration, a system is provided for determining a treatment plan for a radiation therapy system. The system includes the radiation therapy system configured with adjustable beam shape settings to adjust a spatial dose delivered to a subject and a computer system. The computer system is configured to determine a three-dimensional region of interest (ROI) in the subject containing a critical structure, divide the ROI into a grid of dose voxels, and model a radiation dose fluence map for the grid of dose voxels as delivered by the radiation therapy system with a plurality of beamlets to create a modeled radiation dose fluence map, each having a beamlet fluence. The computer system is further configured to determine a voxel-based fluence map optimization (FMO) model for the modeled radiation dose fluence map, determine a conic optimization formulation for the FMO model to create a determined optimization solution, generate an optimized fluence map by updating the modeled radiation dose fluence map with the determined conic optimization solution, and determine the beam shape settings for the optimized fluence map. The system also includes a display configured to display the optimized fluence map.

In yet another configuration, a method is provided for determining a treatment plan for a radiation therapy system. The method includes dividing a three-dimensional volume of a patient into a grid of dose voxels, wherein at least a portion of said dose voxels are designated to belong to at least one target or to at least one critical structure. The method also includes modeling an ionizing radiation dose as delivered by a plurality of beamlets each having a beamlet fluence, providing a quadratic, power and exponential cone voxel-based model for optimizing a fluence map, said fluence map defining said fluences for each of said plurality of beamlets, and solving said model based on defined treatment plan clinical objectives and constraints for said at least one target and said at least one critical structure using an interior point algorithm to obtain a globally optimal fluence map.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention. Like reference numerals will be used to refer to like parts from Figure to Figure in the following description.

### BRIEF DESCRIPTION OF THE DRAWINGS

FIG. 1 is a block diagram of a non-limiting example image-guided radiation therapy (“IGRT”) system that may be used in accordance with the present disclosure.

FIG. 2 is a block diagram of a control system that may be configured according to the present disclosure to control a radiation treatment system.

FIG. 3 is a flowchart of non-limiting example steps for a—method of fluence map optimization.

FIG. 4 shows graphs of non-limiting example survival fraction approximation results.

FIG. 5 shows graphs of non-limiting example seriality in NTCP approximation results.

### DETAILED DESCRIPTION

Systems and methods are provided for conic optimization of FMO with commonly used treatment plan criteria. Conic optimization is a structured approach to mathematical optimization. Modeling FMO problems as conic optimization problems enables the use of advanced numerical solvers for FMO problems, and results in faster and better solutions to FMO problems than conventional methods for FMO.

Formulating FMO problems in conic form includes many advantages, such as that the conic optimization framework is very general and can provide for many FMO problem forms. The conic form also provides for optimal solutions and enables the use of advanced numerical solvers that are more readily available than conventional systems.

Many dose-based and biologically-based treatment planning criteria can be (re)formulated as convex objectives and constraints. In accordance with the present disclosure, fluence map optimization problems can be formulated as convex optimization problems. The main advantage of convex optimization problems is that any local optimum is a global optimum.

Interior point methods (IPMs) are general purpose algorithms for constrained convex optimization. They guarantee optimality of the found solution and are efficient in most cases. Moreover, and in particular for primal-dual implementations, IPMs are regarded as highly efficient in practice in the optimization community.

Conic optimization allows for non-linear evaluation criteria, and is thus more general than linear optimization. Non-linear evaluation criteria may be formulated by restricting variables to belong to certain convex ‘cones’. By restricting the types of allowed cones, an optimization may be more structured than general convex optimization. Conic optimization provides for optimization that is more structured and less error prone. As an example, it is not necessary to specify derivatives and Hessians, as would be required in conventional convex optimization approaches.

Highly efficient general purpose primal-dual IPMs exist for conic optimization using the linear, quadratic, exponential and power cone. It is not necessary to develop and maintain a solver for FMO problems of a specific form. General purpose solvers guarantee to solve the conic form problems to optimality in polynomial time. This provides a theoretical backing that other approaches lack. Previous solutions, such as iCycle, have been tailored for optimization problems with a specific functional form. That is, its interior-point implementation accepts particular evaluation criteria, along with their gradients and Hessians. Inclusion of additional criteria is not straightforward.

In some configurations, conic optimization problems can be solved by efficient general purpose conic solvers. Next to flexibility and modeling benefits, problems formulated as conic optimization problems can benefit from improvements in current conic solvers as well as other developments in conic optimization research.

The generality of conic optimization carries advantages for biologically-based FMO problems. The potential of biological-based models over surrogate dose-based criteria has been recognized, but practical implementation presents many challenges.

In accordance with the present disclosure, a conic optimization methodology for FMO is provided with a focus on biological-based evaluation criteria. In some configurations, only the linear, quadratic, exponential and power cone may be used in the optimization. FMO problems may be formed as conic optimization problems, and general purpose conic solvers may be used in the optimization.

Conic representations are provided for all common biological-based treatment plan evaluation criteria, e.g., TCP, NTCP and gEUD criteria, making use of the linear, quadratic, power and exponential cone. New treatment plan evaluation criteria can be incorporated in a similar manner, if conic representable.

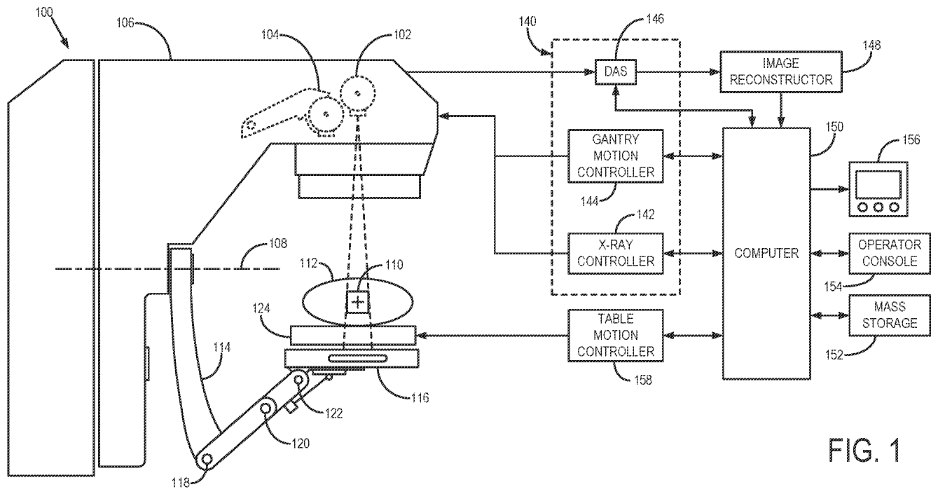

Referring to FIG. 1, an exemplary image-guided radiation therapy (“IGRT”) system 100 that may be used in accordance with the present disclosure includes a therapeutic x-ray source 102 and a diagnostic x-ray source 104. The diagnostic x-ray source 104 projects a cone-beam of x-rays toward a detector array 116. Both the therapeutic x-ray source 102 and diagnostic x-ray source 104 are attached adjacent each other and housed at the same end of a first rotatable gantry 106, which rotates about a pivot axis 108. The first rotatable gantry 106 allows either of the x-ray sources, 102 and 104, to be aligned in a desired manner with respect to a target volume 110 in a subject 112 positioned on a patient table 124. A second rotatable gantry 114 is rotatably attached to the first rotatable gantry 106 such that it too is able to rotate about the pivot axis, 108. Disposed on one end of the second rotatable gantry 114 is an x-ray detector 116. The x-ray detector 116 functions not only as a diagnostic image device when receiving x-rays from the diagnostic x-ray source 104, but also as a portal image device when receiving x-rays from the therapeutic x-ray source 102. The detector array 116 is formed by a number of detector elements that together sense the projected x-rays that pass through the subject 112. Each detector element produces an electrical signal that represents the intensity of an impinging x-ray beam and, hence, the attenuation of the beam as it passes through the subject 112. The second rotatable gantry 114 further includes an articulating end that can pivot about three points 118, 120, and 122. The pivoting motion provided by these points 118, 120, and 122, allows the x-ray detector 116 to be moved within a two-dimensional plane.

The rotation of the rotatable gantries, 106 and 114, and the operation of the x-ray sources, 102 and 104, are governed by a control mechanism 140 of the IGRT system. The control mechanism 140 includes an x-ray controller 142 that provides power and timing signals to the x-ray sources, 102 and 104, and a gantry motor controller 144 that controls the rotational speed and position of the gantries, 106 and 114. A data acquisition system (“DAS”) 146 in the control mechanism 140 samples analog data from detector elements and converts the data to digital signals for subsequent processing. An image reconstructor 148, receives sampled and digitized x-ray data from the DAS 146 and performs high speed image reconstruction. The reconstructed image is applied as an input to a computer 150 which stores the image in a mass storage device 152.

The computer 150 also receives commands and scanning parameters from an operator via a console 154 that has a keyboard. An associated display 156 allows the operator to observe the reconstructed image and other data from the computer 150. The operator supplied commands and parameters are used by the computer 150 to provide control signals and information to the DAS 146, the x-ray controller 142 and the gantry motor controller 144. In addition, the computer 150 operates a table motor controller 158 which controls the motorized patient table 124 to position the subject 112 within the gantries, 106 and 114.

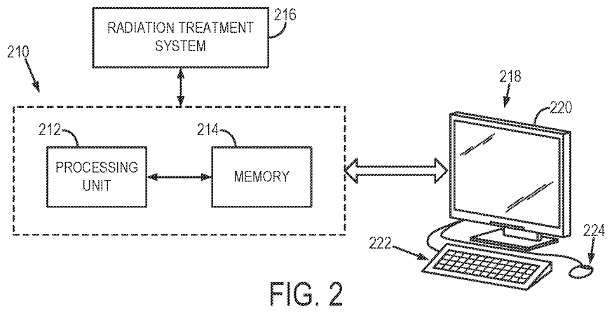

Referring to FIG. 2, a control system 218 that may be configured according to the present disclosure to control a radiation treatment system 216 is shown. Control system 218 may include a user display 220, interface systems 222 and 224. In a non-limiting example, interface system 222 and 224 may include a keyboard and a mouse as shown. The control system 218 may receive commands or control inputs through interface system 222 and 224, and may display reports or results, such as a report of an optimized fluence map on the user display 220.

Control system 218 may also include processing capability 210, which may include a processor 212 and a memory 214. The processor 212 may be configured to determine an optimized fluence map, or otherwise perform a conic optimization as described below. Memory 214 may store the optimized fluence map, or may store instructions for controlling the radiation treatment system 216 using an optimized fluence map.

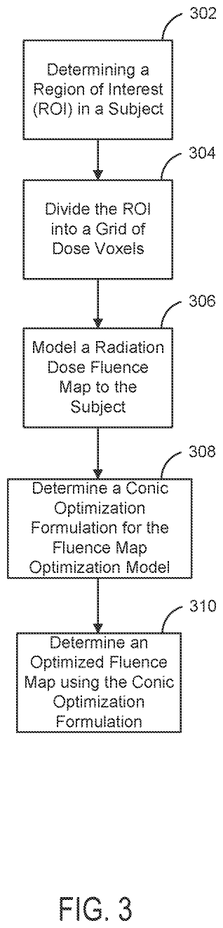

Referring to FIG. 3, a flowchart of non-limiting example steps for a method of fluence map optimization is shown. A region of interest (ROI) may be determined for a subject at step 302. The ROI may include at least one target for radiotherapy or at least one critical structure. A critical structure may include a target for radiotherapy, such as a tumor, or may include a critical organ near a target where a goal may be to avoid exposing the critical structure to damaging radiation. The ROI may include a 3D volume within the subject.

The ROI may be divided into a grid of dose voxels at step 304. In some configurations, dividing a three-dimensional ROI volume of a subject into a grid of dose voxels includes designating a portion of the dose voxels to belong to at least one target or to at least one critical structure. The grid may be generated by any appropriate means, such as by placing a Cartesian grid in the ROI, or by designating a select voxel size to make up the ROI, or the like.

A model of a radiation dose fluence map to the subject may be created at step 306. Modeling a fluence map may include modeling an ionizing radiation dose as delivered by a plurality of beamlets from a radiation therapy system, each beamlet having a beamlet fluence. Modeling may include any appropriate modeling method, such as a computer simulation, an estimation of a fluence map, and the like. A fluence map may also be created from a previously-acquired tracking of a radiation delivery, such as a poral imaging system that tracks the fluence map from a previous radiation delivery.

A conic optimization formulation may be determined for the fluence map at step 308. A conic optimization formulation may include using a quadratic, power or exponential cone in a voxel-based model for optimizing a fluence map. The FMO model may include defining fluences for each of a plurality of beamlets for the radiation therapy system. In some configurations, conic optimization includes an interior point convex optimization and an optimized fluence map may be generated by determining a treatment plan clinical objective and constraints for the ROI of the subject using an interior point convex optimization.

An optimized fluence map may be determined at step 310 based upon the conic optimization formulation. In some configurations, conic optimization may include using an interior point convex optimization with at least one of a primal-dual or barrier interior point solver. In some configurations, conic optimization includes applying convex functions selected as at least one of piece-wise linear functions, convex nonlinear functions, or piece-wise non-linear convex functions. The optimization may also be performed in polynomial time to provide for a fast solution that can be deployed to a radiation therapy system in a rapid period of time.

### Conic Optimization Methodology

Conic optimization may be understood starting from linear optimization. Let c∈ [symbol] ⁿ, A∈ [symbol] ^{m×n}, b∈ [symbol] ^{m}. A linear optimization problem in standard form may be given by:

$${min}_{x} {c}^{⊤} x \qquad ( 1 )$$

$$s . t . Ax = b$$

$$x ∈ {ℝ}_{{+}^{′}}^{n}$$

where the set [symbol] ₊ⁿ is a non-negative orthant. When moving from linear to convex optimization, an approach may be to replace the objective by a convex function ƒ₀(x) and replace the constraints by convex constraints ƒⱼ(x)≤uⱼ, j=1, . . . , p, where u∈ [symbol] ^{p} is an upper bound vector. A general convex optimization problem may be:

$${min}_{x} {f}_{0} ( x ) \qquad ( 2 )$$

$$s . t . {f}_{j} ( x ) \underline{<} {u}_{j} , j = 1 , … , p .$$

Conic optimization takes an approach where eq. (1) may partially be replaced by a proper cone. A set C is a cone if x∈C implies that λx∈C for all scalars λ≥0. A proper cone is defined as follows.

Definition 1: A cone C⊆ [symbol] ⁿ is a proper cone if it is closed, has a nonempty interior and x∈C, −x∈C⇒x=0 (Pointed); x,y∈C⇒x+y∈C (Convex).

The standard form of a conic optimization problem is:

$${min}_{x} {c}^{⊤} x \qquad ( 3 a )$$

$$s . t . Ax = b \qquad ( 3 b )$$

$$x ∈ C , \qquad ( 3 c )$$

With C a proper cone. The nonnegative orthant [symbol] ₊ⁿ is a proper cone. Another way of moving from linear optimization to conic optimization is by considering the inequality associated with [symbol] ₊ⁿ. The element-wise inequality x≥y is equivalent to xᵢ−yᵢ≥0, i=1, . . . , n, which can also be written as x−y∈ [symbol] ₊ⁿ. Similarly, for a proper cone C the generalized inequality x≥_{c}y is equivalent to x−y≥_{c}0, which is again equivalent to x −y∈C. Conic optimization in itself is not any more restrictive than general convex optimization. Any general convex optimization problem of form (2) can be cast in the conic optimization form (3). The advantages of conic optimization become clear when we restrict ourselves to a few families of proper cones.

Instead of the standard form (3) we consider the general conic optimization form

$${min}_{x} {c}^{⊤} x \qquad ( 4 a )$$

$$s . t . {l}^{c} \leq A x \leq {u}^{c} \qquad ( 4 b )$$

$${l}^{x} \leq x \leq {u}^{x} \qquad ( 4 c )$$

$$x ∈ C , \qquad ( 4 d )$$

with l^{c}, u^{c} bounds on the linear constraints and l^{x}, u^{x} bounds on variable x. Additionally, we assume there exists a partitioning x=(x¹, . . . , x^{p}), such that x^{k}∈Cₖ, i.e., each subvector x^{k} belongs to a smaller cone Cₖ. If C₁, . . . , Cₚ are proper cones, their Cartesian product C=C₁× . . . ×Cₚ is a proper cone. Models may involve a mix of various different proper cones and can be represented in form (4).

In some configurations, a subset of variables belongs to one cone, but this is not a restriction because duplicates of variables can be introduced via linear equality constraints.

### Examples of Practically Useful Cones

Conventionally, IPMs for conic optimization were restricted to the linear cone (non-negative orthant), the quadratic cone and the semidefinite matrix cone. The n-dimensional linear cone may be given by

[symbol]

₊ⁿ={x∈ [symbol] ⁿ:x₁·, xₙ≥0,} (5)

In form (4), the linear cone is redundant because all linear components are captured by (4b) and (4c). The n-dimensional quadratic cone is given by:

Qⁿ={x∈ [symbol] ⁿ:x₁≥{square root over (x₂²+x₃²+ . . . +xₙ²})} (6)

The n-dimensional rotated quadratic cone representation is given by:

Qᵣⁿ={x∈ [symbol] ⁿ:2x₁x₂≥x₃²+ . . . +xₙ², x₁, x₂≥0} (7)

A transformation may be used from quadratic cones to rotated quadratic cones. The linear and quadratic cone are so-called symmetric cones, and offer great modeling flexibility. In some aspects, however, they do not admit the modeling of expressions involving some exponentials and logarithms. Also, many power functions can be represented using quadratic cones, but these functions are more naturally represented using different cones. Also, the semidefinite cone is a third often used symmetric cone, but may have less use for modeling.

In non-limiting examples, two non-symmetric cones are the exponential cone and the power cone. The 3-dimensional exponential cone is given by:

Kₑₓₚ={(x₁,x₂,x₃):x₁≥x₂ exp(x₃/x₂),x₂>0}∪{(x₁,0,x₃):x₁≥0,x₃≤0} (8)

Various representations of the power cone exist. With 0<α<1, one form of the n-dimensional power cone is given by:

[symbol]

ₙ^{α,1-α}={x∈ [symbol] ⁿ:x₁^{α}x₂^{1-α}≥\sqrt{x₃²+ . . . +xₙ²},x₁,x₂≥0}, (9)

A more general version may be represented where the left hand side consists of arbitrary many terms. These four cones, together with the semidefinite cone, can model almost all convex functions arising in practice. Many (mixed-integer) convex problems can be modeled using the linear, quadratic, exponential, and power cone.

Several different IPM approaches exist for solving conic optimization problems with linear, quadratic, exponential and power cones. Barrier methods have been shown to converge in polynomial time for these cones. As an alternative to the (primal) barrier methods, primal-dual IPMs for the symmetric cones may be used. They proved to be efficient and may outperform the barrier methods in practice.

In some configurations, conic optimization using linear, quadratic, exponential and power cones may include disciplined convex programming (DCP). DCP is a modeling framework for convex optimization that uses solely expressions formed from an atom library and a ruleset. The atom library contains simple expressions known to be convex. The DCP ruleset indicates how atoms can be combined into new expressions that preserve convexity. Optimization problems constructed using DCP are convex and conically representable by construction. The MOSEK modeling cookbook provides an overview of (simple) functions and sets that can be represented using the linear, quadratic, exponential and power cone. With the appropriate ruleset, one can also construct more advanced expressions that are conic representable by construction, similar to DCP. Alternatively, the ‘natural’ form of treatment plan evaluation criteria may be used in FMO problems, and their conic representations may be found.

### Conic Reformulations

Reformulating a convex optimization problem P of form (2) to an equivalent problem Q of form (4) typically requires the introduction of auxiliary variables. Let x∈ [symbol] ⁿ denote the variable vector of problem P, and let (x,y)∈ [symbol] ^{n+q} denote the variable vector of problem Q. That is, in Q we distinguish between original variables x that already exist in the original problem P and auxiliary variables y∈ [symbol] ^{q}. Equivalence of P and Q may be defined as follows.

Definition 2: Optimization problems P and Q are equivalent if x* is optimal to P if there exists a y*∈ [symbol] ^{q} such that (x*,y*) is optimal to Q. The idea is that after the reformulation the new problem Q can be represented using only the cones introduced above. A conic problem may be defined as follows.

Definition 3: An optimization problem Q of form (4) is named a conic problem (CP) if all cones Cₖ, k=1, p are linear, quadratic, rotated quadratic, exponential or power cones.

We are now equipped to reformulate problems to equivalent CPs. We start with the functional form (2), as many optimization problems arising in practice come in this form. We rewrite (2) to

$${min}_{x , τ} τ \qquad ( 10 a )$$

$$s . t . {f}_{0} ( x ) \leq t \qquad ( 10 b )$$

$${f}_{j} ( x ) \underline{<} {u}_{j} , j = 1 , … , p , \qquad ( 10 c )$$

where in the FMO setting functions ƒⱼ could be TCP or NTCP functions. This form suggests the analysis of epigraph sets

epi(ƒ)={(x,t):x∈ [symbol] ⁿ,t∈ [symbol] ,ƒ(x)≤t}, (11)

for ƒ₀ and ƒⱼ for all j. We make use of the following definitions:

Definition 4: A constraint is a conic constraint if it is a linear constraint or a constraint of the form x∈C, with C a linear, quadratic, rotated quadratic, exponential or power cone.

Definition 5: A set S⊆ [symbol] ⁿ is conic representable (Cr) if there exists a set T∈ [symbol] ^{n+q} such that T can be described by finitely many conic constraints, and x∈S if there exists an u∈ [symbol] ^{q} such that (x,u)∈T. The conic constraints describing set T are the conic representation (CR) of S.

If, via the introduction of auxiliary variables and constraints, CRs are found for the epigraphs of ƒ₀ and ƒⱼ, j=1, . . . , p, the resulting optimization problem may be a CP that is equivalent to (10) or (2). Considering the increasing functions g₀: [symbol] → [symbol] and gⱼ: [symbol] → [symbol] , j=1, p. Applying go to (10b) is equivalent to

g₀(ƒ₀(x))≤τ (12a)

τ≤g₀(t), (12b)

Where τ∈ [symbol] is a new variable. The objective of (10) is to minimize t, which is equivalent to minimizing g₀(t) because g₀ is increasing. Variable t occurs only in constraint (12b), so τ may be minimized and variable t may be removed. Stated differently, when applying g₀ to both sides of the inequality ƒ₀(x)≤t, the right-hand side g₀(t) may be replaced by new variable τ. Hence, x is optimal to (2) if there exists a τ∈ [symbol] such that (x,τ) is optimal to

$${min}_{x , τ} τ \qquad ( 13 a )$$

$$s . t . {g}_{0} ( {f}_{0} ( x ) ) \underline{<} τ \qquad ( 13 b )$$

$${g}_{j} ( {f}_{j} ( x ) ) \leq {g}_{j} ( {u}_{j} ) , j = 1 , … , p , \qquad ( 13 c )$$

where gⱼ may be applied to (10c). gⱼ(uⱼ) is constant for all j=1, . . . , p. Problem (13) is equivalent to problem (10). This shows that, in order to find a CP equivalent to (2), it suffices to find for every j=0, . . . , p an increasing function gⱼ for which we can find a CR of the set epi(gⱼ∘ƒⱼ). In particular, any function ƒ is suitable for conic optimization if we can find an increasing function g such that epi(g∘ƒ) is Cr.

In the above, an upper bound may be imposed or the function ƒ may be minimized. In case of a lower bound or maximization, consider the hypograph:

hypo(ƒ)={(x,t):x∈ [symbol] ⁿ,t∈ [symbol] ,ƒ(x)≥t}, (14)

Function ƒ is suitable for conic optimization if an increasing function g can be found such that hypo(g∘ƒ) is Cr.

### Conic Representations of Common Evaluation Criteria

A generic FMO problem has the form:

$${min}_{x , D} {f}_{0} ( {D}^{0} ) \qquad ( 15 a )$$

$$s . t . {f}_{j} ( {D}^{j} ) \leq {u}_{j} , j = 1 , … , p \qquad ( 15 b )$$

$$Ax = D \qquad ( 15 c )$$

$$x \geq 0. \qquad ( 15 d )$$

The goal is to minimize a particular treatment plan evaluation criteria ƒ₀, subject to constraints on evaluation criteria ƒ₁, . . . , ƒₚ. Treatment criteria j=0, . . . , p may be evaluated in dose vector D^{j}. All dose vectors may be stacked in D. Constraint (15c) relates D to the pencil beam weights x according to pencil-beam (or dose-deposition) matrix A. Constraint (15d) ensures (elementwise) nonnegativity of pencil beam weights.

Commonly used objectives and constraints in radiation therapy treatment optimization can be reformulated to conic form. According to the methodology above, for each function ƒ that is to be minimized or has an upper bound, a strictly increasing function g can be found and a CR of epi(g∘ƒ) if ƒ. Alternatively, if ƒ is to be maximized or has a lower bound, a strictly increasing function g and a CR of hypo(g∘ƒf) can be found.

In some configurations, a radiation therapy optimization problem consisting only of functions that can be reformulated to conic form can be solved in polynomial-time via primal-dual IPMs. In a non-limiting example, a set of n voxels indexed by i may be used. Let D∈ [symbol] ₊ⁿ denote the voxel doses. Let vᵢ>0 denote the relative volume of voxel i, i.e., Σᵢ₌₁ⁿv₁=1. For every voxel i dose Dᵢ is delivered uniformly over N fractions, with dᵢ denote the dose per fraction, i.e., dᵢ=Dᵢ/N. Let α>0 denote the tissue radiation sensitivity parameter and let α/β>0 denote the tissue fractionation sensitivity parameter.

### The Survival Fraction Function

The LQ survival fraction function gives the surviving fraction of cells in voxel I after receiving a dose of Dᵢ as:

$$S {F}_{L Q} ( {D}_{i} ) = exp \{ - α {D}_{i} ( 1 + \frac{1}{α / β} \frac{{D}_{i}}{N} ) \} . \qquad ( 16 )$$

Eq. (16) is convex, as long as the voxel dose satisfies

$${D}_{i} \geq \sqrt{\frac{1}{2} \frac{( α / β ) N}{α}} - \frac{1}{2} ( α / β ) N . \qquad ( 17 )$$

$$L = max \{ 0 , \sqrt{\frac{1}{2} \frac{( α / β ) N}{α}} - \frac{1}{2} ( α / β ) N \} \qquad ( 18 )$$

A bound of Dᵢ≥L may be considered and is not overly restrictive.

If α/β→∞ or if N=1 the quadratic term in the survival fraction function (16) vanishes. In this case, the survival fraction function reduces to the single-hit model:

SF_{L}(Dᵢ)=exp{−αDᵢ} (19)

The inequality SF_{L}(D)≤t is equivalent to the system of conic constraints

$$\{ \begin{matrix}( t , 1 , x ) ∈ {K}_{exp} & & & & & & & & & & & & & & ( 20 a ) \\ x = - α D . & & & & & & & & & & & & & & ( 20 b )\end{matrix}$$

A strictly increasing function g may not be required for the reformulation, so g(t)=t may be selected. Then epi(g∘SF_{L}) is Cr. In the general case (with fractionation), the LQ survival function may only be convex on a subset (i.e., Dᵢ≥L) of its natural domain. Such functions are typically not Cr. In these situations, an approximation may be used.

With reference to SCM ten Eikelder, A Ajdari, T Bortfeld, D den Hertog, “Conic formulation of fluence map optimization problems,” Phys. Med. Biol. 66 225016, (Nov. 24, 2021), let D̅≥L and

[symbol]

(Dᵢ;D̅)=max {0,a₁(D̅)Dᵢ+a₂(D̅)}exp{a₃(D̅)Dᵢ+a₄(D̅)}, (21)

With fitting parameters a₁(D̅), a₃(D̅)<0, a₂(D̅)>0, a₄(D̅). Then [symbol] (Dᵢ;D̅)≤SF_{LQ}(Dᵢ) holds for all Dᵢ≥L, and [symbol] (Dᵢ;D̅) is tangent to SF_{LQ}(D) at Dᵢ=D̅.

A non-limiting example approximation may be found by taking the pointwise maximum over [symbol] (Dᵢ;D̅) for various values of D̅. Numerical results may be used to demonstrate the approximation quality.

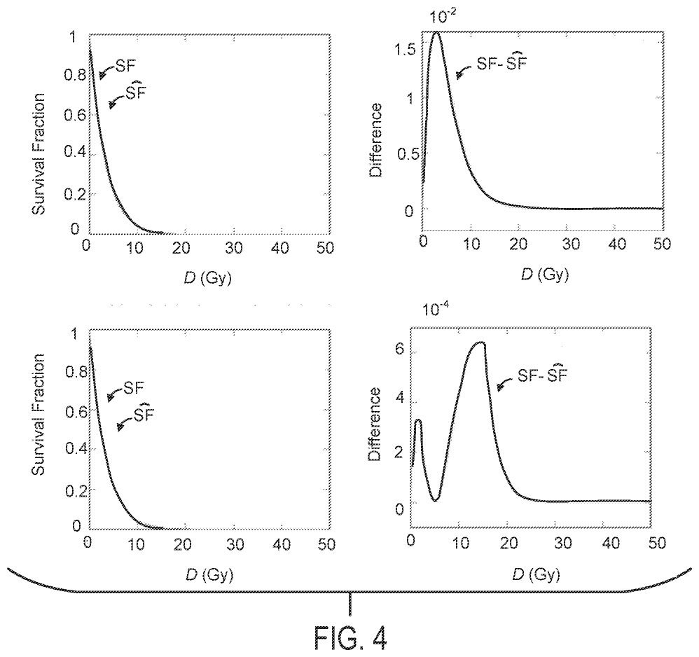

Referring to FIG. 4, graphs of non-limiting example survival fraction approximation are shown. The graphs (top row) depict a comparison of survival fraction function SF_{LQ}(D) and approximation [symbol] (D,30), with α=0.3, α/β=10, N=30, where the tangent point D̅=30. The maximum difference was attained at D=5. An improved approximation can be obtained by adding D̅=5 as a second tangent point. The approximation function is the pointwise maximum of [symbol] (D,5) and SF_{LQ}(D,30). As shown (bottom row), using two tangent points yields a very accurate approximation.

The epigraph of (21) is Cr. Consider the inequality [symbol] (D);D̅)≤t. An auxiliary variable y∈ [symbol] may be used to eliminate the max operator multiplied by a₃(D̅)/a₁(D̅). Then the inequality is equivalent to:

$$\{ \text{} \begin{matrix}\frac{{a}_{1} ( \overline{D} )}{{a}_{3} ( \overline{D} )} y exp & \{ y - \frac{{a}_{2} ( \overline{D} ) {a}_{3} ( \overline{D} )}{{a}_{1} ( \overline{D} )} + {a}_{4} ( \overline{D} ) \} \leq t & & & ( 22 a ) \\ y \geq {a}_{3} ( \overline{D} ) {D}_{i} + & \frac{{a}_{2} ( \overline{D} ) {a}_{3} ( \overline{D} )}{{a}_{1} ( \overline{D} )} & & & ( 22 b ) \\ y \geq 0. & & & & ( 22 c )\end{matrix}$$

Subsequently, because the constant in front of the term y exp{y} is positive, this term can be extracted using an auxiliary variable z∈ [symbol] and rewrite (22a) to

$$\{ \begin{matrix}\frac{{a}_{1} ( \overline{D} )}{{a}_{3} ( \overline{D} )} exp & \{ - \frac{{a}_{2} ( \overline{D} ) {a}_{3} ( \overline{D} )}{{a}_{1} ( \overline{D} )} + {a}_{4} ( \overline{D} ) \} z \leq t & & & & & & ( 23 a ) \\ y exp \{ y \} \leq z & & & & & & & ( 23 b )\end{matrix}$$

Because y≥0 the inequality (23b) can be written as

$$y exp ( {y}^{2} / y ) \leq z ⇔ \{ \begin{matrix}y exp ( w / y ) \leq z \\ {y}^{2} \leq w\end{matrix} ⇔ \{ \begin{matrix}( z , y , w ) ∈ {K}_{exp} \\ ( 1 / 2 , w , y ) ∈ {Q}_{r}^{3}\end{matrix} . \qquad ( 24 )$$

Putting everything together, [symbol] (Dᵢ;y)≤t holds if there exist scalar variables y, z, w such that

$$\{ \begin{matrix}\frac{{a}_{1} ( \overline{D} )}{{a}_{3} ( \overline{D} )} exp \{ - \frac{{a}_{2} ( \overline{D} ) {a}_{3} ( \overline{D} )}{{a}_{1} ( \overline{D} )} + {a}_{4} ( \overline{D} ) \} z \leq t & ( 25 a ) \\ ( z , y , w ) ∈ {K}_{exp} & ( 25 b ) \\ ( 1 / 2 , w , y ) ∈ {𝒬}_{r}^{3} & ( 25 c ) \\ y \geq {a}_{3} ( \overline{D} ) {D}_{i} + \frac{{a}_{2} ( \overline{D} ) {a}_{3} ( \overline{D} )}{{a}_{1} ( \overline{D} )} & ( 25 d ) \\ y \geq 0. & ( 25 e )\end{matrix}$$

This methodology does not require a strictly increasing function g for the reformulation. Instead, if g(t)=t, then epi(g∘ [symbol] ) is Cr on Dᵢ≥L.

### The Linear-Quadratic (LQ) TCP Function

With the survival fraction function (21), the LQ based TCP is given by

TCP_{LQ}(D)=exp(−N₀Σᵢ₌₁ⁿvᵢ [symbol] (Dᵢ), (26)

where N₀ is the total initial number of clonogenic cells. The total number of tumor cells remaining (TNTCR) is given by −log(TCP_{LQ}), which gives

TNTCR_{LQ}(D)=N₀Σᵢ₌₁ⁿvᵢ [symbol] (Dᵢ) (27)

Generally the goal is to maximize TCP, a CR of hypo (g∘TCP_{LQ}) may be found and the inequality TCP_{LQ}(D)≥t may be considered, for some t∈[0,1). Considering the strictly increasing function g(t)=log(t), and let τ denote either the new objective variable or the constant term that replaces g(t), then, with y∈ [symbol] ⁿ an auxiliary variable, TCP_{LQ}(D)≥t is equivalent to

$$\{ \begin{matrix}- {N}_{0} {\sum }_{i = 1}^{n} {v}_{l} {y}_{i} \geq τ & ( 28 a ) \\ {y}_{i} \geq ( {D}_{i} ) , ∀ i . & ( 28 b )\end{matrix}$$

The second inequality is Cr. Without fractionation, the linear TCP can be reformulated analogously, where [symbol] in eq. (28b) is replaced by SF_{L} (see eq. (19)). With p∈ [symbol] an auxiliary variable, TNTCR_{L}(D)≤t can be reformulated to

$$\{ \begin{matrix}{N}_{0} {\sum }_{i = 1}^{n} {v}_{i} exp \{ - α {D}_{i} - p \} \leq 1 & ( 29 a ) \\ p \leq log ( t ) , & ( 29 b )\end{matrix}$$

With the introduction of auxiliary variables y, z∈ [symbol] ⁿ, the first inequality is equivalent to

$$\{ \begin{matrix}{N}_{0} {\sum }_{i = 1}^{n} {v}_{i} {y}_{i} \leq 1 & ( 30 a ) \\ {y}_{i} \geq exp \{ {z}_{i} \} , ∀ i & ( 30 b ) \\ {z}_{i} = - α {D}_{i} - p , ∀ i . & ( 30 c )\end{matrix}$$

A benefit of this reformulation is that the exponents zᵢ have smaller absolute values than −αDᵢ itself if p<0 (i.e., upper bound t≤1), thus improving numerical stability.

The logarithmic tumor control probability (LTCP), which is a variation on the linear TCP may be given by:

$$LTCP ( D ) = \frac{1}{n} {\sum }_{i = 1}^{n} exp \{ - α ( {D}_{i} - {D}_{p} ) \} , \qquad ( 31 )$$

With α the radiation sensitivity parameter and Dₚ the prescribed dose. It can be reformulated to conic form similar to the linear TCP function.

### Equivalent Uniform Dose (EUD)

The EUD of a nonuniform dose to the tumor is the dose that, if delivered homogeneously to the tumor, yields the same TCP. With the single-hit TCP model the EUD given by:

$${EUD}_{L} ( D ) = - \frac{1}{α} log ( \frac{1}{n} {\sum }_{i = 1}^{n} {SF}_{L} ( {D}_{i} ) ) . \qquad ( 32 )$$

In some configurations, an objective may be to maximize tumor EUD, where the inequality EUD_{L}(D)≥t may be considered. With strictly increasing function g(t)=−exp{−αt} and g(t) replaced by new variable τ, hypo(g∘EUD_{L}) is equivalent to:

$$\{ \begin{matrix}\frac{1}{n} {\sum }_{i = 1}^{n} {y}_{i} \leq - τ & ( 33 a ) \\ {y}_{i} \geq {SF}_{L} ( {D}_{i} ) , ∀ i , & ( 33 b )\end{matrix}$$

The latter inequality is Cr.

For the general case, with survival function [symbol] , the EUD is given by

$${EUD}_{LQ} ( D ) = - \frac{1}{2} \frac{α}{β} [ 1 - \sqrt{1 - \frac{4 β}{{α}^{2} N} log ( \frac{1}{n} {\sum }_{i = 1}^{n} ( {D}_{i} ) )} ] \qquad ( 34 )$$

The inequality EUD_{LQ}(D)≥t. By extracting the survival fraction functions and rewriting, this is equivalent to

$$\{ \begin{matrix}- \frac{1}{n} {\sum }_{i = 1}^{n} {y}_{i} \geq - exp \{ - α N ( t + \frac{1}{α / β} {t}^{2} ) \} & ( 35 a ) \\ {y}_{i} \geq ( {D}_{i} , ∀ i . & ( 35 b )\end{matrix}$$

With a strictly increasing function

$$g ( t ) = - exp \{ - α N ( t + \frac{1}{α / β} {t}^{2} ) \}$$

and g(t) replaced by new variable τ, hypo(g∘EUD_{LQ}) is equivalent to

$$\{ \begin{matrix}- \frac{1}{n} {\sum }_{i = 1}^{n} {y}_{i} \geq τ & ( 36 a ) \\ {y}_{i} \geq ( {D}_{i} ) , ∀ i . & ( 36 b )\end{matrix}$$

The generalized equivalent uniform dose (gEUD) of a heterogeneous dose distribution is given by:

$$gEUD ( D ; a ) = {( \frac{1}{n} {\sum }_{i = 1}^{n} {D}_{i}^{a} )}^{\frac{1}{a}} , \qquad ( 37 )$$

where α is a tissue specific parameter. As a→+∞, the gEUD approaches the maximum dose; as a→+∞, the gEUD approaches the minimum dose. a=1 yields the mean dose and as a→0 the gEUD approaches the geometric mean. For OARs, it may be assumed that a≥1 and for tumor it may be assumed that a>0. (37) may be rewritten to:

$$gEUD ( D ; a ) = {{n}^{- \frac{1}{a}} ( {\sum }_{i = 1}^{n} {D}_{i}^{a} )}^{\frac{1}{a}} = {n}^{- \frac{1}{a}} {‖ D ‖}_{a} , \qquad ( 38 )$$

where ∥⋅∥ₐ is the generalized a−norm. a<0 and a≥ 1 are explored below.

For (a<0), and for a tumor volume, the inequality ∥D∥ₐ≥t may be used. This is equivalent to t≥Σᵢ₌₁ⁿDᵢ^{a}t^{1-a}, which, on its part, is equivalent to:

$$\{ \begin{matrix}{\sum }_{i = 1}^{n} {y}_{i} \leq t & ( 39 a ) \\ {y}_{i}^{\frac{1}{1 - a}} {D}_{i}^{\frac{- a}{1 - a}} \geq t , ∀ i & ( 39 b ) \\ {D}_{i} \geq 0 , ∀ i . & ( 39 c )\end{matrix}$$

Eq. (39b) is equivalent to

$$( {y}_{i} , {D}_{i} , t ) ∈ {𝒫}_{3}^{\frac{1}{1 - a} , \frac{- a}{1 - a}} , ∀ i , \qquad ( 40 )$$

The hypograph of the gEUD function with a<0 can be modeled using a power cone. Eq. (39b) is conic quadratic representable (CQr) if a<0 is restricted to be a rational number. For the reformulation methodology, g(t)=t and the above result shows that hypo(g∘gEUD) is Cr for a<0.

The fractionation-corrected version, i.e., ∥BED(D)∥ₐ≥t is Cr. It reduces to

$$\{ \begin{matrix}{‖ y ‖}_{a} \geq t & ( 42 a ) \\ BED ( {D}_{i} ) \geq {y}_{i} , ∀ i , & ( 42 b )\end{matrix}$$

Optimization using only the linear and quadratic cone is known as conic quadratic programming or second-order cone programming, but the latter constraint is non-convex.

For (a>1), and for an organ-at-risk (OAR), the inequality ∥D∥ₐ≤t may be used. This can be rewritten to Σᵢ₌₁ⁿDᵢ^{a}t^{1-a}≤t. Subsequently, this is equivalent to

$$\{ \begin{matrix}{\sum }_{i = 1}^{n} {y}_{i} \leq t & ( 43 a ) \\ {D}_{i} \leq {y}_{i}^{\frac{1}{a}} {t}^{\frac{a - 1}{a}} , ∀ i & ( 43 b ) \\ {D}_{i} \geq 0 , ∀ i . & ( 43 c )\end{matrix}$$

The inequality in eq. (43b) is equivalent to:

$$( {y}_{i} , t , {D}_{i} ) ∈ {𝒫}_{3}^{\frac{1}{a} , \frac{a - 1}{a}} , ∀ i , \qquad ( 44 )$$

The epigraph of the gEUD function with a≥1 can be modeled using a power cone. Similar to the previous case, if a≥1 is restricted to be a rational number, eq. (43b) can again be shown to be CQr. For the reformulation methodology, g(t)=t and the above result shows that epi(g∘gEUD) is Cr for a≥1.

To account for non-standard fractionation schemes, a Biological Effective Dose (BED) model may be used. If voxel i receives a dose Dᵢ, its BED is given by

$$BED ( {D}_{i} ) = {D}_{i} ( 1 + \frac{D}{( α / β ) N} ) . \qquad ( 41 )$$

Let BED (D) denote the BED vector associated with dose vector D. The fractionation-corrected version of ∥D∥ₐ≤t, i.e., ∥BED(D)∥ₐ≤t is CQr. It reduces to

$$\{ \begin{matrix}{‖ y ‖}_{a} \leq t & ( 45 a ) \\ BED ( {D}_{i} ) \leq {y}_{i} , ∀ i . & ( 45 b )\end{matrix}$$

The latter constraint is equivalent to:

$${D}_{i} + \frac{{D}_{i}^{2}}{( α / β ) N} \leq {y}_{i} ⇔ \{ \begin{matrix}{D}_{i} + \frac{1}{( α / β ) N} {z}_{i} \leq {y}_{i} \\ {D}_{i}^{2} \leq {z}_{i}\end{matrix} ∀ i . \qquad ( 46 )$$

The latter inequality can be expressed as (zᵢ,½,Dᵢ)∈Qᵣ³ for all i.

### Normal Tissue Complication Probability (NTCP) Functions

NTCP functions can be given by

$${NTCP}_{LKB} ( D ) = Φ ( \frac{D - {D}_{50}}{{mD}_{50}} ) , \qquad ( 47 )$$

where D is a homogeneous dose and

$$Φ ( z ) = 1 / \sqrt{2 π} {\int }_{- ∞}^{z} {e}^{- \frac{1}{2} {x}^{2}} dx$$

is the standard normal cumulative distribution function (CDF). Parameter D₅₀ represents the dose that yields a 50% complication probability and m>0 is a parameter for the slope of the NTCP curve. The inequality NTCP_{LKB}(D)≤t can be rewritten as

$$\frac{D - {D}_{50}}{{mD}_{50}} \leq {Φ}^{- 1} ( ) t ) ⇔ D \leq {D}_{50} ( 1 + m {Φ}^{- 1} ( t ) ) , \qquad ( 48 )$$

Which constitutes a linear constraint. Function Φ⁻¹ is a strictly increasing function, and so is g(t)=D₅₀(1+mΦ⁻¹(t)). With τ a new variable taking the value g(t), epi(g∘NTCP_{LKB}) is equivalent to D≤τ. The inequality is linear, so Cr.

The NTCP LKB function may be applied to the gEUD of a heterogeneous dose D. With the same strictly increasing function g, the epigraph epi(g∘NTCP_{LKB}) is equivalent to gEUD(D;a)≤τ, where τ again replaces g(t). As shown, gEUD constraints are Cr.

Mechanistic concepts may be used to derive a phenomenological NTCP model:

$${NTCP}_{A \& N} ( D ; a ) = 1 - exp \{ - {( \frac{gEUD ( D \cdot a )}{Δ} )}^{a} \} , \qquad ( 49 )$$

Where a≥1 is the gEUD parameter and Δ is the dose that yields a 1−e⁻¹ complication probability. The inequality NTCP_{A&N}(D;a)≤t can be rewritten as

$$gEUD ( D ; a ) \leq Δ {log ( \frac{1}{1 - t} )}^{\frac{1}{a}} \qquad ( 50 )$$

Hence, with a strictly increasing function

$$g ( t ) = Δ {log ( \frac{1}{1 - t} )}^{\frac{1}{a}} ,$$

the epigraph epi(g∘NTCP_{A&N}) is equivalent to gEUD(D;a)≤τ, where τ replaces g(t). This is again Cr.

For the NTCP models above, the fractionation-corrected version is also Cr. The inequality NTCP(BED(D))≤t is equivalent to:

$$\{ \begin{matrix}NTCP ( z ) \leq t & ( 51 a ) \\ BED ( {D}_{i} ) \leq {z}_{i} , ∀ i , & ( 51 b )\end{matrix}$$

With the latter is Cr according to (46).

The relative-seriality s-model may be given by:

$${NTCP}_{RS} ( D ) = {( 1 - {\prod }_{i = 1}^{n} {( 1 - exp {\{ - {N}_{0} {SF}_{LQ} ( {D}_{i} ) \}}^{s} )}^{{v}_{i}} )}^{1 / s} \qquad ( 52 )$$

Parameter s∈(0,1] is the relative seriality parameter. An approximation for the CR of the epigraph of NTCP_{RS}(D) may be found.

With reference to SCM ten Eikelder, A Ajdari, T Bortfeld, D den Hertog, “Conic formulation of fluence map optimization problems,” Phys. Med. Biol. 66 225016, (Nov. 24, 2021), for D∈ [symbol] ⁿ such that 0≤Dᵢ≤U, i=1, n, define:

$$N {RS}_{} ( D ) = {( 1 - {\prod }_{i = 1}^{n} exp \{ {v}_{i} {min}_{k - 1 , … , K} {h}_{k} ( α BED ( {D}_{i} ) ) \} )}^{1 / s} \qquad ( 53 )$$

with linear functions hₖ:[0,U]→ [symbol] , k=1 , . . . , K . Then NTCP_{RS}(D)≤N [symbol] _{RS}(D).

This provides a conservative approximation to the relative seriality NTCP model. The minimum over functions hx constitutes a piecewise-linear approximation.

Similar to the approximation for the survival fraction function (Item [00124]), adding more points to set K improves the approximation. Numerical results demonstrating the approximation quality can be performed.

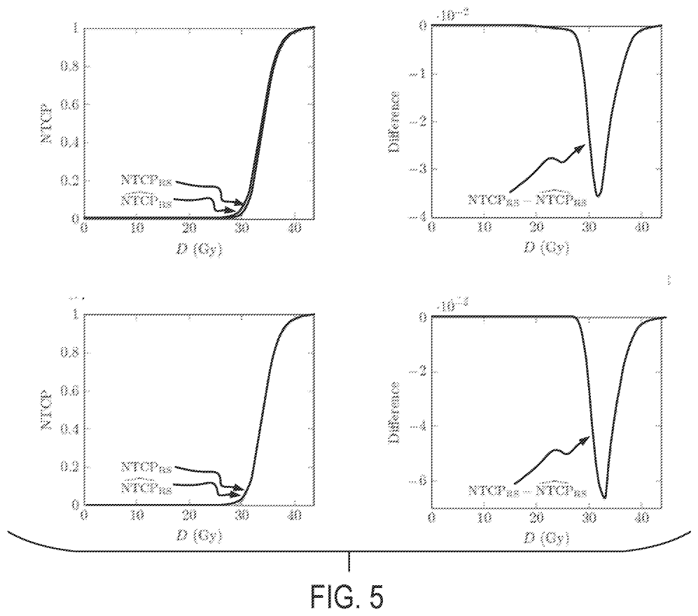

Referring to FIG. 5, graphs of non-limiting example seriality in NTCP approximation are shown. The relative seriality NTCP function as depicted in eq. (52) is multivariate. A dose vector D∈[30,40]ⁿ, with n=100,000 voxels, vᵢ=1/n for i=1, . . . , n may be uniformly sampled. Let L=0, U=50, α=0.3, α/β=3, N₀=10₆ and s=0.2. By scaling dose vector D to a given mean dose D, the NTCP curve can be visualized. FIG. 5 shows NTCP_{RS} and the approximation NTCP_{RS} with the dose vector D scaled according to the horizontal axis. In order to have scaled voxel doses in the interval [0,50], mean dose is restricted to [0, 43.75].

It can be shown that eq. (53) is Cr. Consider the inequality N [symbol] _{RS}(D)≤t. The strictly increasing function g(t)=−log(1−t^{s}) on both sides may be used to obtain:

$$- {\sum }_{i = 1}^{n} {v}_{i} {min}_{k = 1 , … , K} {h}_{k} ( α BED ( {D}_{i} ) ) \leq - log ( 1 - {t}^{s} ) . \qquad ( 54 )$$

The auxiliary variable τ may be introduced with substitution τ=g(t). Subsequently, both sides may be multiplied by −1 and an auxiliary variable y∈ [symbol] ⁿ may be used to obtain:

$$\{ \begin{matrix}{\sum }_{l = 1}^{n} {v}_{i} {y}_{i} \geq - τ & ( 55 a ) \\ {y}_{i} \leq {h}_{k} ( α B E D ( {D}_{i} ) ) , ∀ k , i . & ( 55 b )\end{matrix}$$

A new variable z∈ [symbol] ⁿ may be used to extract the CQr BED term and find:

$$\{ \begin{matrix}{\sum }_{i = 1}^{n} {v}_{i} {y}_{i} \geq - τ & ( 56 a ) \\ {y}_{i} \leq {h}_{k} ( {z}_{i} ) , ∀ k , i & ( 56 b ) \\ {z}_{i} \geq α BED ( {D}_{i} ) , ∀ i . & ( 56 c )\end{matrix}$$

Because the functions hₖ, k=1, . . . K, are linear, eq. (56) is a CR of epi(g∘NTCP_{RS}).

### Sigmoidal Criteria Based on gEUD

A sigmoidal-shaped function may be maximized based on the gEUD for both target volume and OARs. With a the gEUD parameter, their logistic function based criteria may be given by:

$$ω ( D ; a ) = \{ \begin{matrix}\frac{1}{1 + {( \frac{{gEUD}_{0}}{gEUD ( D ; a )} )}^{k}} & if a < 0 \\ \frac{1}{1 + {( \frac{gEUD ( D ; a )}{{gEUD}_{0}} )}^{k}} & if a < 1 ,\end{matrix} \qquad ( 57 )$$

Where the target volume gEUD₀ is the gEUD of the prescription dose. For OARs gEUD₀ is the gEUD of the tolerable uniform dose, e.g., D₅₀. Parameter k>0 determines the steepness of the function at gEUD₀. To maximize function w, the inequality w(D;a)≥t, with t∈(0,1) may be used. If a<0, this reduces to:

$$gEUD ( D ; a ) \geq {{gEUD}_{0} ( \frac{t}{1 - t} )}^{\frac{1}{k}} , \qquad ( 58 )$$

The right-hand-side (RHS) is strictly increasing in t for t∈(0,1). The RHS may be denoted by g(t) and replaced by a new variable τ. Then the hypograph hypo(g∘w) is equivalent to gEUD(D)≥τ. This is Cr according. If a≥1, we obtain

$$gEUD ( D ; a ) \geq - {{gEUD}_{0} ( \frac{1 - t}{t} )}^{\underline{1} / k} , \qquad ( 59 )$$

The RHS is strictly increasing in t for t∈(0,1). The RHS may be denoted by g(t) and replaced by a new variable τ. The hypograph hypo(g∘w) is equivalent to gEUD(D)≤−τ. This is Cr.

In some configurations, a sigmoidal-shaped function may be maximized based on the CDF of the normal distribution. The following criteria may be obtained:

$$\breve{ω} ( D ; a ) = \{ \begin{matrix}1 - Φ ( \frac{{gEUD}_{0} - gEUD ( D ; α )}{σ {gEUD}_{0}} ) & if a < 0 \\ 1 - Φ ( \frac{gEUD ( D ; α ) - {gEUD}_{0}}{σ {gEUD}_{0}} ) & if a \geq 1 ,\end{matrix} \qquad ( 60 )$$

Where σ determines the steepness at gEUD₀. The interest may be in the inequality w(D;a)≥t, t∈(0,1). In case a<0, the inequality reduces to:

gEUD(D;a)≥gEUD₀(1−σΦ⁻¹(1−t)), (61)

The RHS is increasing in t for t∈(0,1). The RHS may be denoted by g(t) and replaced by a new variable τ. The hypograph hypo(g∘w) is equivalent to gEUD(D;a)≥τ which is Cr for a<0. Similarly, for a≥1 and strictly increasing function g(t)=−gEUD₀(1+σΦ⁻¹(1−t)), the hypograph hypo(g∘w) is equivalent to gEUD(D;a)≤−τ.

### Prescription Dose Based Functions

A general prescription dose-based function for tumors may be given as:

ƒ_{T}(D)=Σᵢ₌₁ⁿvᵢ max{0,pᵢ−Dᵢ}^{γ}, (62)

where pᵢ is the prescription dose to voxel i, γᵢ≥1 is the shape parameter of the error and vᵢ is the relative volume of voxel i. Common choices γ=1 and γ=2 correspond to absolute errors and squared errors, respectively. A goal may be to minimize this error, and as such the inequality ƒ_{T}(D)≤t. By extracting the max operator this can be rewritten to:

$$\{ \begin{matrix}{\sum }_{i = 1}^{n} {v}_{i} {y}_{i}^{γ} \underline{<} t & ( 63 a ) \\ {y}_{i} \geq max \{ 0 , {p}_{i} - {D}_{i} \} , ∀ i , & ( 63 b )\end{matrix}$$

which is equivalent to:

$$\{ \begin{matrix}{\sum }_{i = 1}^{n} {v}_{i} {z}_{i} \leq t & ( 64 a ) \\ {z}_{i} \geq {y}_{i}^{γ} , ∀ i & ( 64 b ) \\ {y}_{i} \geq {p}_{i} - {D}_{i} , ∀ i & ( 64 c ) \\ {y}_{i} \geq 0 , ∀ i . & ( 64 d )\end{matrix}$$

If γ=1, then the second inequality is linear. If γ>1, because yᵢ≥0, the second inequality is equivalent to zᵢ^{γ}^{ᵢ}−≥yᵢ, which yields (zᵢ,1,yᵢ)∈ [symbol] ₃^{1/γ,1-1/γ}. Therefore, all inequalities in eq. (64) are conic constraints. With g(t)=t, the epigraph epi(g∘ƒ_{T}) is Cr.

In a non-limiting example, the prescription dose-based function for OARs may be given by:

ƒ_{OAR}(D)=Σᵢ₌₁ⁿvᵢ max{0,Dᵢ−pᵢ}^{γ} (65)

For OARs it is common that pᵢ=0 for all voxels. The reformulation of ƒ_{OAR} is analogous to that of ƒ_{T}.

### Quadratic Smoothing Constraints

Smoothing constraints may be used to prevent spiked beamlet profiles, and generate smoother fluence maps that are more easily deliverable in practice using multileaf collimators, and are less sensitive to patient movement. The second derivative of the fluence may be used as an indication of smoothness. This may be discretized and the quadratic smoothness term may be defined as:

$${s}_{q} ( x ) = \frac{1}{2} {x}^{T} {S}_{{q}^{X}} , \qquad ( 66 )$$

With S_{q}∈ [symbol] ^{n×n} a symmetric positive definite matrix. Matrix F∈ [symbol] ^{p×n} may be such that S_{q}=F^{τ}F, e.g., and F can be the Cholesky factor of A. A new variable y∈ [symbol] ⁿ may be used to extract the linear term Fx. Then S_{q}(x)≤t is equivalent to

$$\{ \begin{matrix}{y}^{T} y \leq 2 t & ( 67 a ) \\ y = Fx & ( 67 b )\end{matrix}$$

The first constraint is equivalent to (t,1,y)∈Qᵣⁿ⁺². Thus, with strictly increasing function g(t)=t,epi(g∘s_{q}) is Cr. If upper bound t is a constant, eq. (67a) can also be rewritten to (\sqrt{2t}, y)∈Qⁿ⁺¹.

An linear form may be given by sₗ(x)=Sₗx, with Sₗ a block diagonal matrix with entries −(1−ϵ), (1−ϵ), −1,0, +1, positioned such that adjacent beamlet intensity variation is at most ϵ. This is Cr with g(t)=t.

Table 1 below provides an overview of non-limiting example results without fractionation. The column ‘type’ indicates min if the epigraph is Cr, and max if the hypograph is Cr (i.e., it can be used for minimization resp. maximization). Table 1 provides an overview of the cones (next to the linear cone) necessary to describe the radiation therapy criteria in a single-hit case (excluding fractionation). For each criterion the strictly increasing function g(t) used in the reformulation is also provided. Column 3 indicates the criterion type; for minimization criteria the epigraph of epi(g∘ƒ) is Cr, for maximization criteria the hypograph hypo(g∘ƒ) is Cr. For cases where the conic reformulation depends on gEUD parameter a, its range is indicated.

TABLE 1
Overview of non-limiting example results without fractionation.

| Criterion | Parameter range | Type | g(t) | Cones* |
|---|---|---|---|---|
| SF_{L}(D) | — | min | t | Kₑₓₚ |
| TCP_{L}(D) | — | max | log(t) | Kₑₓₚ |
| EUD_{L}(D) | — | max | −exp(−αt) | Kₑₓₚ |
| gEUD(D; a) | a < 0 | max | t | [symbol] ₃ |
| | a ≥ 1 | min | t | [symbol] ₃ |
| NTCP_{LKB}(D) | — | min | D₅₀(1 + mΦ⁻¹(t)) | none |
| NTCP_{LKB}(gEUD(D; a)) | a ≥ 1 | min | D₅₀(1 + mΦ⁻¹(t)) | [symbol] ₃ |
| NTCP_{A&N}(D; a) | a ≥ 1 | min | $Δ {log ( \frac{1}{1 - t} )}^{\frac{1}{a}}$ | [symbol] ₃ |
| w(D; a) | a < 0 | max | ${{gEUD}_{0} ( \frac{t}{1 - t} )}^{\frac{1}{k}}$ | [symbol] ₃ |
| | a ≥ 1 | max | $- {{gEUD}_{0} ( \frac{t}{1 - t} )}^{\frac{1}{k}}$ | [symbol] ₃ |
| [symbol] w(D; a) | a < 0 | max | gEUD₀(1 − σΦ⁻¹(1 −t)) | |
| | a ≥ 1 | max | −gEUD₀(1 + σΦ⁻¹(1 − t) | [symbol] ₃ |
| f_{T}(D) | — | min | t | [symbol] ₃ |
| f_{OAR}(D) | — | min | t | [symbol] ₃ |
| s_{q}(x) | — | min | t | Q [symbol] |
| sₗ(x) | — | min | t | none |

*next to the linear cone [symbol]
indicates data missing or illegible when filed

Table 2 below provides an overview of non-limiting example results incorporating fractionation via the linear-quadratic model. For the LQ survival fraction model eq. (16) and the relative seriality NTCP model eq. (52) the conic reformulations are approximate. The survival fraction model is also used for the LQ TCP model eq. (26) and the LQ EUD model eq. (34). The column ‘type’ indicates min if the epigraph is Cr, and max if the hypograph is Cr (i.e., it can be used for minimization resp. maximization).

TABLE 2
overview of non-limiting example results incorporating
fractionation via the linear-quadratic model.

| | Parameter | | | |
|---|---|---|---|---|
| Criterion | range | Type | g(t) | Cones* |
| [symbol] _{LQ}(D) | — | min | t | Kₑₓₚ; Qᵣ³ |
| TCP_{LQ}(D) | — | max | log(t) | Kₑₓₚ; Qᵣ³ |
| EUD_{LQ}(D) | — | max | $exp ( - α N ( t + \frac{{t}^{2}}{α / β} ) )$ | Kₑₓₚ; Qᵣ³ |
| gEUD(BED(D); a) | a ≥ 1 | min | t | [symbol] ₃, Qᵣ³ |
| NTCP_{LKB} (BED(D)) | — | min | D₅₀(1 + mΦ⁻¹(t)) | Qᵣ_{³} |
| NTCP_{LKB}(BED(gEUD(D; a))) | a ≥ 1 | min | D₅₀(1 + mΦ⁻¹(t)) | [symbol] ₃, Qᵣ³ |
| NTCP [symbol] (BED(D)) | a ≥ 1 | min | $Δ {log ( \frac{1}{1 - t} )}^{\frac{1}{a}}$ | [symbol] ₃, Qᵣ³ |
| [symbol] _{RS}(D) | — | min | −log(1 − t [symbol] ) | Qᵣ³ |

*Next to the linear cone. [symbol]
indicates data missing or illegible when filed

### Multi-Criteria Optimization

The conic reformulation methodology may be used for weighted sums and products of multiple criteria. In some configurations, the epigraph of the sum of two functions need not be Cr if the individual epigraphs are Cr, and thus the products of TCP functions or sigmoidal functions may be challenging to add to the library above. However, in FMO weighted sums and products are often encountered as part of a multi-criteria optimization (MCO) approach. Typically, several criteria are not specified as hard constraints but are considered objectives, and in treatment planning a trade-off has to be made between these conflicting objectives. By choosing appropriate criteria weights, the relative importance of different evaluation criteria can be quantified in the objective of the FMO problem.

MCO may be used for handling such trade-offs, and may be used to compute a set of solutions known as the Pareto surface (or frontier). This is the set of all solutions for which the objective value of one objective function cannot be strictly decreased without strictly increasing the objective value of another objective function. It may be regarded as the set of all meaningful candidate solutions that could be considered in decision making. There are many methods that may be used for generation of the Pareto surface, such as scalarization methods. In a non-limiting example, the scalarization method may be a weighted sum method, which introduces a weighting vector λ∈ [symbol] ₊^{p} and solves for objectives ƒ₁, . . . , ƒₚ the problem:

$${min}_{D ∈ S} {\sum }_{j = 1}^{p} {λ}_{j} {f}_{j} ( D ) \qquad ( 68 )$$

With set S representing additional constraints on dose vector D. By varying the weight vector λ, a database of treatment plans may be created.

Product criteria, such as composite TCP terms, can be cast as weighted sum criteria. In a non-limiting example, with [symbol] a set of target volumes, the problem

$${max}_{D ∈ S} {\prod }_{s ∈ 𝒯} T C {{P}_{s} ( {D}_{s} )}^{{λ}_{s}}$$

is equivalent to the problem

$${max}_{D ∈ S} {\sum }_{s ∈ 𝒯} {λ}_{S} log ( {TCP}_{s} ( {D}_{S} ) ) .$$

Here, log(⋅) may be a strictly increasing function. Both weighted sums and weighted products can be seen as instances of an MCO procedure.

Another non-limiting example MCO method is the E-constraint method, which introduces a vector ε∈ [symbol] ^{p} and solves for each k=1, . . . , p the problem:

$${min}_{D ∈ S} {f}_{k} ( D ) \qquad ( 69 a )$$

$$s . t . {f}_{j} ( D ) \leq {ε}_{j} , j = 1 … , p , j \neq k . \qquad ( 69 b )$$

By varying the λ vector in eq. (68) resp. the minimization criteria k in eq. (79), the Pareto surface can be computed. The E-constraint method solves problems with a single objective function, so if CRs of the epigraphs of the individual treatment criteria are found, the E-constraint method can be used in the conic reformulation methodology to obtain the Pareto surface.

Systems and methods in accordance with the present disclosure have been provided for reformulating FMO problems to conic optimization problems. Treatment plan evaluation criteria may be conic representable, thus making the methodology generally applicable.

The present disclosure has described one or more preferred embodiments, and it should be appreciated that many equivalents, alternatives, variations, and modifications, aside from those expressly stated, are possible and within the scope of the invention.

1. A method of determining a treatment plan for a radiation therapy system, comprising:
determining a three-dimensional region of interest (ROI) in a subject containing a critical structure;
dividing the ROI into a grid of dose voxels;
modeling a radiation dose fluence map for the grid of dose voxels as delivered by the radiation therapy system with a plurality of beamlets to create a modeled radiation dose fluence map, each having a beamlet fluence;
determining a voxel-based fluence map optimization (FMO) model for the modeled radiation dose fluence map;
determining a conic optimization formulation for the FMO model to create a determined conic optimization solution;
generating an optimized fluence map by updating the modeled radiation dose fluence map with the determined conic optimization solution.

2. The method of claim 1, wherein the conic optimization is a convex optimization that provides a globally optimal fluence map.

3. The method of claim 2, wherein the voxel-based FMO model includes at least one of a quadratic, power, or exponential cone.

4. The method of claim 1, wherein the conic optimization uses an interior point method and wherein generating the optimized fluence map includes determining a treatment plan with clinical objective and constraints for the ROI.

5. The method of claim 4, wherein the interior point method includes at least one of a primal-dual or barrier interior point solver.

6. The method of claim 4, wherein determining the conic optimization formulation includes applying convex functions selected as at least one of piece-wise linear functions, convex nonlinear functions, or piece-wise non-linear convex functions.

7. The method of claim 4, wherein the interior point method includes determining the conic optimization solution in polynomial time.

8. The method of claim 4, wherein the constraints include quadratic smoothing.

9. The method of claim 1, wherein determining the conic optimization formulation includes determining an approximation that is conically representable for a clinical objective or constraint.

10. The method of claim 1, wherein the ROI includes at least one of a radiation therapy target or a critical structure.

11. A system for determining a treatment plan for a radiation therapy system, comprising:
the radiation therapy system configured with adjustable beam shape settings to adjust a spatial dose delivered to a subject;
a computer system configured to:
i) determine a three-dimensional region of interest (ROI) in the subject containing a critical structure;
ii) divide the ROI into a grid of dose voxels;
iii) model a radiation dose fluence map for the grid of dose voxels as delivered by the radiation therapy system with a plurality of beamlets to create a modeled radiation dose fluence map, each having a beamlet fluence;
iv) determine a voxel-based fluence map optimization (FMO) model for the modeled radiation dose fluence map;
v) determine a conic optimization formulation for the FMO model to create a determined optimization solution;
vi) generate an optimized fluence map by updating the modeled radiation dose fluence map with the determined conic optimization solution;
vii) determine the beam shape settings for the optimized fluence map; and
a display configured to display the optimized fluence map.

12. The system of claim 11, wherein the conic optimization is a convex optimization that provides a globally optimal fluence map.

13. The system of claim 12, wherein the voxel-based FMO model includes at least one of a quadratic, power, or exponential cone.

14. The system of claim 11, wherein the computer system is further configured to determine the conic optimization solution with an interior point method and wherein the computer system is further configured to determine treatment plan clinical objectives and constraints for the ROI and generate the optimized fluence map by using an interior point method.

15. The system of claim 14, wherein the interior point method includes at least one of a primal-dual or barrier interior point solver.

16. The system of claim 14, wherein the computer system is further configured to determine the conic optimization formulation by applying convex functions selected as at least one of piece-wise linear functions, convex nonlinear functions, or piece-wise non-linear convex functions.

17. The system of claim 14, wherein the computer system is further configured to determine the conic optimization solution in polynomial time via interior point methods.

18. The system of claim 14, wherein the constraints include quadratic smoothing.

19. The system of claim 11, wherein the computer system is further configured to determine the conic optimization formulation by determining an approximation that is conically representable for a clinical objective or constraint.

20. The system of claim 11, wherein the ROI includes at least one of a radiation therapy target or a critical structure.

21. A method of determining a treatment plan for a radiation therapy system, the radiation therapy system comprising a radiation therapy unit, wherein a spatial dose delivered can be changed by adjusting beam shape settings, the method including steps comprising:
dividing a three-dimensional volume of a patient into a grid of dose voxels, wherein at least a portion of said dose voxels are designated to belong to at least one target or to at least one critical structure;
modeling an ionizing radiation dose as delivered by a plurality of beamlets each having a beamlet fluence;
providing a quadratic, power and exponential cone voxel-based model for optimizing a fluence map, said fluence map defining said fluences for each of said plurality of beamlets; and
solving said model based on defined treatment plan clinical objectives and constraints for said at least one target and said at least one critical structure using an interior point algorithm to obtain a globally optimal fluence map.

22. The method of claim 21, wherein the interior point method comprises a primal-dual or barrier interior point solver.

23. The method of claim 21, wherein providing the conic formulation comprises applying convex functions selected from the group consisting of piece-wise linear functions, convex nonlinear functions, and piece-wise non-linear convex functions.

24. The method of claim 21, wherein the interior point method ensures solving the conic form optimization problem in polynomial time.

25. The method of claim 21, wherein the constraints include quadratic smoothing.

26. The method of claim 21, further comprising controlling the radiation therapy unit at least based on the globally optimal fluence map.
